(19) United States
(12) Patent Application Publication
(10) Pub. No.: US 2020/0085604 A1
(43) Pub. Date: Mar. 19, 2020

(54) Orthosis, In Particular Orthosis For Gonarthrosis

(71) Applicant: THUASNE Deutschland GmbH, Burgwedel (DE)

(72) Inventors: Herbert SCHÖBEL, Burgwedel (DE); Nicolas VIOLEAU, La-Valla-en-Gier (FR); Romain PANNETIER, Lyon (FR)

(21) Appl. No.: 16/467,889

(22) PCT Filed: Dec. 5, 2017
(86) PCT No.: PCT/EP2017/081549

§ 371 Date: Jun. 7, 2019

(30) Foreign Application Priority Data
Dec. 7, 2016 (DE) 20 2016 106 817.7

(51) Int. Cl. A61F 5/01

(52) U.S. Cl. CPC A61F 5/0109

(57) ABSTRACT

An orthosis, particularly an orthosis for gonarthrosis, for relieving the load on the medial or lateral knee compartment of the human knee. Orthosis includes a belt, system, and the belt system has one or more stable-tension belt devices. Each belt device includes one or more end belts. Further, each belt device has one or more connecting belts which are angularly adjustably connected to each end belt of the belt device.

### BRIEF DESCRIPTION OF THE INVENTION

The invention relates to an orthosis, in particular an orthosis for gonarthrosis, for relieving the stress on the medial or lateral knee compartment of the human knee.

Orthotic aids assist the function of joints and are used for stabilizing, immobilizing and/or relieving the stress on the corresponding joint in order to stabilize and guide the joint in the case of illness or injury, for example, with the result that a correct and pain-free position is established and fixed.

An orthosis of the type in question is known from EP 2 246 014 B1. The orthosis comprises a belt system comprising two adjustable belts which are fastened to a flexible cuff and are arranged in a criss-cross manner. Relief of stress of the knee joint, in the case of knee problems, can be achieved by means of the orthosis. The belts ensure that a joint line opens up when the knee joint is opened, such that, depending on the arrangement of the orthosis, stress of either the medial knee compartment or of the lateral knee compartment is relieved in a therapeutic manner, and thus pain-free movement is achieved.

The belts are intended to apply forces lateral or medially, via a pressure point, so as to be as central in the leg as possible, at the level of the compromise pivot axis of the knee. It may be the case, however, that the pressure point is actually arranged too far in the dorsal or distal direction, i.e. more in the region of the back of the knee. An incorrect arrangement of this kind means that high forces have to be applied, using the belts, in order to achieve sufficient relief of stress of the knee. The region of the back of the knee is sensitive to pressure, and therefore the incorrect arrangement and the high tension of the belts may lead to pain when wearing the orthosis. Moreover, the movement of the knee may be impeded. Overall, the wearing comfort of the orthosis described in EP 2 246 014 B1 may be reduced.

The object of the invention is therefore that of providing an orthotic aid, in particular an orthosis for gonarthrosis, the wearing comfort of which is improved compared with the orthoses in the prior art.

The present invention therefore relates to an orthosis for relieving the stress on the medial or lateral knee compartment of the human knee, comprising a belt system, the belt system comprising at least one tension-resistant belt device. In this case, each belt device comprises at least one end belt and at least one connection belt in each case, which connection belt is connected to each end belt of the relevant belt device so as to be adjustable with respect to the angle. As a result, it is possible to set an angle in the course of the belt device and to position a pressure point. Overall, anatomical belt guidance is thus possible. In a preferred embodiment, the orthosis is an orthosis for gonarthrosis.

In one embodiment, the belt system comprises a criss-cross guide for two belt devices, in which the two belt devices cross over one another in the region of the criss-cross guide and are retained in a positive manner by means of the criss-cross guide.

In one embodiment, the at least one end belt of at least one belt device consists in each case of exactly one proximal end belt of the relevant belt device and exactly one distal end belt of the relevant belt device. In this case, the connection belt of the relevant belt device comprises a proximal and a distal end region. The proximal end region connects the relevant connection belt to the proximal end belt of the relevant belt device. The distal end region connects the relevant connection belt to the distal end belt of the relevant belt device.

In one embodiment, at least one end belt of at least one belt device is connected to the connection belt of the relevant belt device, by means of a hook and loop connection, so as to be adjustable with respect to the angle and so as to be detachable.

In a further embodiment, the connection belt of the relevant belt device comprises a belt element having a front face and a rear face. At each end, on the rear face, the belt element in each case comprises a hook strip for the hook and loop connection, and a foldable tab that is arranged so as to correspond to the hook strip and comprises a further hook strip facing the hook strip. The end belts comprise a loop strip on either side, at least in portions, which loop strip can be positioned between the hook strips. The end belts can be connected to the connection belt so as to be adjustable with respect to the angle, by means of the hook and loop connection that is provided by the hook strips and the loop strips.

In one embodiment, at least one end belt of at least one belt device is connected to the relevant connection belt of the relevant belt device, so as to be adjustable with respect to the angle, by means of a screw connection or by means of a rivet connection. In a preferred embodiment, for the purpose of length adjustment the at least one end belt of at least one belt device comprises a lower belt portion and an upper belt portion which is wrapped around on the lower belt portion, a length-adjustable loop being formed by the belt portions.

In a further embodiment, the belt device comprises a quick-release fastener on the upper belt portion of the end belt, by means of which fastener the loop can be releasably closed. In a preferred embodiment, the quick-release fastener may comprise a magnet that magnetically pulls the quick-release fastener into a closed position and locks the fastener in the closed position.

In one embodiment of the present invention, an end of the end belt that is arranged on the upper belt portion is fastened, by means of a hook and loop connection, in a region that can be selected for length adjustment of the loop.

In an embodiment according to the invention, the orthosis comprises a cuff to which the belt system is fastened. In this case, the at least one belt device consists of exactly one first belt device comprising a first connection belt, and exactly one second belt device comprising a second connection belt, the two belt devices crossing over one another and each being fastened at a proximal region and at a distal region of the cuff. The belt system further comprises fastening devices comprising cords, by means of which the two belt devices are fastened to the cuff. In addition, return eyelets are provided on the belt system, on which eyelets the end belts of the belt devices are retained, each cord of each fastening device in each case passing through a return eyelet and retaining the return eyelet on the relevant fastening device so as to have one translational degree of freedom and two rotational degrees of freedom. In a preferred embodiment, the at least one fastening device is fastened to the cuff so as to be vertically displaceable. In a particularly preferred embodiment, the at least one fastening device comprises a base part on which each cord of the relevant fastening device is retained at two vertically mutually spaced points in each case and, together with the base part, forms a loop in each case. In one embodiment, the base part may comprise a hook strip and the cuff may comprise a loop strip at least in portions. The hook strip and the loop strip together form a hook and loop connection, by means of which the base part can be fastened and positioned on the cuff.

In one embodiment, points are arranged on the base part, at which points a cord of a fastening device is retained, and which points are in each case paired with the points at which the other cord of the fastening device is retained. In a preferred embodiment, each cord of at least one fastening device is guided through the base part, proceeding from one of the vertically mutually spaced points in each case. In the process, each cord is deflected in the base part by an angle of between 80° and 100° and, in order to tension the relevant belt device, one end of the cord is guided out of the base part. In this case, the relevant fastening device comprises a grip part that is assigned to the base part and that is connected to the ends of the cords of the relevant fastening device that are guided out of the base part.

The present invention is characterized by the embodiments in the claims, and will be described in greater detail by way of the embodiments in the following description and the figures.

### DESCRIPTION OF THE FIGURES

FIG. 1: shows a first detail of an orthosis according to the invention, comprising two criss-cross connection belts that are each connected to two end belts so as to be adjustable with respect to the angle.

FIG. 2: shows a second detail of the orthosis according to the invention from FIG. 1, comprising a fastening device for two end belts.

FIG. 3: shows a second detail from FIG. 1, of an orthosis according to the invention according to a second embodiment of the invention, comprising an alternative fastening device for two end belts.

FIG. 4: is a first view (for the entire proximal-distal extension) of the orthosis according to the invention of FIG. 1 and FIG. 2.

FIG. 5: is a second view (for the entire proximal-distal extension) of the orthosis according to the invention of FIG. 1, FIG. 2 and FIG. 4.

### DETAILED DESCRIPTION OF THE INVENTION

The present invention relates to an orthotic aid, in particular an orthosis (1), which can preferably be used for treating gonarthrosis.

In the case of an orthosis comprising a belt system (2) in which the belt system (2) comprises at least one tension-resistant belt device (3, 4), each belt device (3, 4) comprising at least one end belt (9, 10, 11, 12) in each case, according to the invention each belt device (3, 4) comprises at least one connection belt (7, 8) in each case, which connection belt is connected to each end belt (9, 10, 11, 12) of the relevant belt device (3, 4) so as to be adjustable with respect to the angle. In a preferred embodiment, exactly one connection belt (7, 8) is provided, which connection belt is connected to each end belt (9, 10, 11, 12) of the relevant belt device (3, 4) so as to be adjustable with respect to the angle. As a result, it is possible to set an angle in the course of the belt device (3, 4) and to position a pressure point. Overall, anatomical belt guidance is thus possible. Any pain when wearing the orthosis is effectively counteracted thereby, and therefore the wearing comfort is greatly increased. The orthosis is preferably an orthosis for gonarthrosis, for relieving the stress on the medial or lateral knee compartment of the human knee.

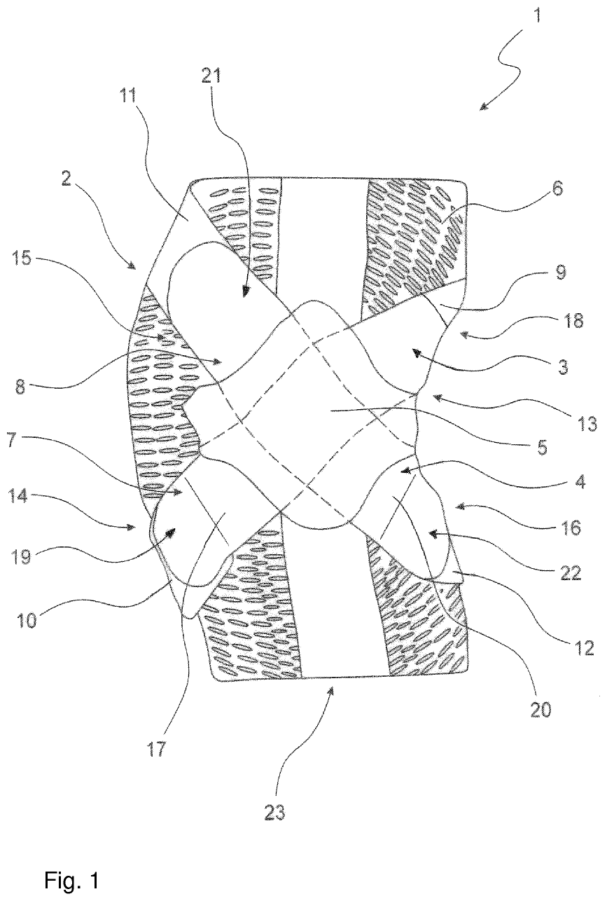

In one embodiment of the orthosis according to the invention, at least one belt device (3, 4) consists of exactly one first belt device (3) comprising a first connection belt (7), and exactly one second belt device (4) comprising a second connection belt (8), as is shown in FIG. 1 for example. The orthosis thus comprises exactly two tension-resistant belt devices (3, 4), by means of which the pressure point is set and by means of which action of force of the orthosis can be adjusted. In this case, each belt device (3, 4) preferably consists, in each case, of the connection belt (7, 8) of the relevant belt device (3, 4) and of at least one end belt (9, 10, 11, 12) of the relevant belt device (3, 4).

In a further embodiment, the belt system (2) comprises a criss-cross guide (5) for two belt devices (3, 4). In this case, the two belt devices (3, 4) cross over one another in the region of the criss-cross guide (5) and are retained in a positive manner by means of the criss-cross guide (5), as is shown for example in FIG. 1. In this case, the belt devices (3, 4) wind in a helical manner around the joint that is to be supported by the orthosis, and bring about advantageous introduction of tensile forces. The pressure point is thus located in the region in which the belt devices (3, 4) cross over one another.

According to one embodiment, as an alternative or in addition to the criss-cross guide (5), the connection belt (7, 8) of at least one belt device (3, 4) comprises two layers, at least in regions. The layers are interconnected at least at two mutually spaced connections. As a result, the layers form a loop or lozenge shape between the connections. In a preferred embodiment, the loops of two connection belts (7, 8) mutually engage. Each loop thus guides at least one layer of the connection belt (7, 8) of a further belt device (3, 4). Accordingly, in a particularly preferred embodiment the belt devices (3, 4) can be displaced relative to one another only within limits specified by the length of the connection belts (7, 8), and are otherwise retained against one another.

The at least one end belt (9, 10, 11, 12) of at least one belt device (3, 4) preferably consists, in each case, of exactly one proximal end belt (9, 11) and exactly one distal end belt (10, 12) of the relevant belt device (3, 4). Accordingly, the first belt device (3) comprises a proximal first end belt (9) and a distal first end belt (10), and the second belt device (4) comprises a proximal second end belt (11) and a distal second end belt (12). Each belt device (3, 4) thus comprises two end belts (9, 10, 11, 12) and a connection belt (7, 8) that is arranged between the end belts and interconnects the end belts (9, 10, 11, 12). In this case, the connection belt (7, 8) of the relevant belt device (3, 4) comprises a proximal end region (13, 15), by means of which the relevant connection belt (7, 8) is connected to the proximal end belt (9, 11) of the relevant belt device (3, 4). The connection belt (7, 8) of the relevant belt device furthermore comprises a distal end region (14, 16), by means of which the relevant connection belt (7, 8) is connected to the distal end belt (10, 12) of the relevant belt device (3, 4). As can be seen in FIG. 1 for example, the first connection belt (7) comprises a proximal first end region (13), by means of which the connection belt is connected to the proximal first end belt (9) so as to be adjustable with respect to the angle and so as to be detachable, and a distal first end region (14), by means of which the connection belt is connected to the distal first end belt 10 so as to be adjustable with respect to the angle and so as to be detachable. In a manner corresponding to the arrangement of the first connection belt (7), the second connection belt (8) comprises a proximal (15) and a distal (16) second end region, by means of which the second connection belt is connected to a proximal second end belt (11) and distal second end belt (12), respectively, so as to be adjustable with respect to the angle and so as to be detachable.

With reference to the invention, specifications such as proximal or distal are used in a manner analogous to specifications with reference to the part of the human body to which the orthosis according to the invention can be applied, in particular the knee joint.

In a preferred embodiment, at least one end belt (9, 10, 11, 12) of at least one belt device (3, 4) is connected to the connection belt of the relevant belt device (3, 4), by means of a hook and loop connection (18, 19, 20, 21), so as to be adjustable with respect to the angle and so as to be detachable. In particular, both end belts of the relevant belt device (3, 4) are connected to a connection belt (7, 8) of the belt device (3, 4) by means of a hook and loop connection (18, 19, 20, 21). This preferably for both belt devices (3, 4), and therefore a hook and loop connection (18, 19, 20, 21) is provided in each case, at each end of each connection belt (7, 8). In the case of two connection belts (7, 8) four hook and loop connections (18, 19, 20, 21) are thus provided, at which connections the angle of the belt devices (3, 4) can be set. The angle adjustment at both ends of the connection belt (7, 8) makes it possible for the angle at which the connection belt (7, 8) extends to remain largely uncoupled from the angle at which the belt device (3, 4) as a whole extends. This is in particular also advantageous in conjunction with the criss-cross guide (5).

The hook and loop connections (18, 19, 20, 21) between the connection belt (7, 8) and the end belts are preferably a double or double-sided hook and loop connection (18, 19, 20, 21) in each case. In this case, the connection belt (7, 8) of the relevant belt device (3, 4) comprises a belt element (17) having a front face and a rear face. At each end, on the rear face the belt element (17) in each case comprises a hook element for the hook and loop connection (18, 19, 20, 21), and a foldable tab that is arranged so as to correspond to the hook element and comprises a further hook strip facing the hook strip. In this case, the end belts (9, 10, 11, 12) comprise a loop strip on either side, at least in portions, which loop strip can be positioned between the hook strips. The loop strip is preferably made of velour. In this case, the end belt (9, 10, 11, 12) comprising the loop strip can optionally be understood to be an end belt having a surface that comprises loops, in particular a velour surface, or an end belt having a strip fastened to the surface thereof, which strip comprises the loops. The end belts (9, 10, 11, 12) can be connected to the connection belt (7, 8) so as to be adjustable with respect to the angle, by means of the hook and loop connection (18, 19, 20, 21) that is provided by the hook strips and the loop strips. The double-sided hook and loop connection (18, 19, 20, 21) provides particular security against inadvertent release of the hook and loop connection.

In one embodiment, as an alternative or in addition to the hook and loop connection (18, 19, 20, 21), at least one end belt of at least one belt device (3, 4) is connected to the connection belt (7, 8) of the relevant belt device (3, 4), so as to be adjustable with respect to the angle, by means of a screw connection.

According to a further embodiment, as an alternative or in addition to the hook and loop connection (18, 19, 20, 21) and/or to the screw connection, at least one end belt (9, 10, 11, 12) of at least one belt device (3, 4) is connected to the connection belt (7, 8) of the relevant belt device (3, 4), so as to be adjustable with respect to the angle, by means of a rivet connection. The rivet connection allows for the angle adjustment, but otherwise holds the connection belt (7, 8) and the end belt (9, 10, 11, 12) against one another, such that it is not possible to adjust the length of the connection belt in the region of the rivet connection.

In contrast, in the case of the hook and loop connection (18, 19, 20, 21) and in the case of the screw connection, it is possible to adjust the length of the belt device (3, 4) in the region of the relevant connection. In order to adapt the length of the at least one belt device (3, 4), different regions of at least one end belt (9, 10, 11, 12) of the relevant belt device (3, 4) can preferably be connected to the connection belt (7, 8) of the relevant belt device (3, 4). Alternatively or in addition, for the purpose of length adjustment an end belt (9, 10, 11, 12) of the relevant belt device (3, 4) can be connected by means of different regions of the connection belt (7, 8) of the relevant belt device (3, 4). A further option for adapting the length of the belt device (3, 4) consists in wrapping a belt portion of the end belt (9, 10, 11, 12). The wrapping leads to the formation of a loop which is of varying lengths depending on the length of the wrapped belt portion. Accordingly, according to a preferred embodiment, for the purpose of length adjustment, at least one end belt (9, 10, 11, 12) of at least one belt device (3, 4) comprises a lower belt portion and an upper belt portion which is wrapped around on the lower belt portion, a length-adjustable loop being formed by the belt portions.

In order to form the loop, the free end of the upper belt portion must be fastened to the lower belt portion. The free end of the upper belt portion is preferably detachably fastened to the lower belt portion in order to apply the orthosis (1) when the belt device (3, 4) is slackened, and to subsequently be able to tauten the belt device (3, 4) at a prespecified length, in an uncomplicated manner. For this purpose, in a preferred embodiment the belt device (3, 4) comprises a quick-release fastener (40) on the upper belt portion of the end belt, by means of which fastener the loop can be releasably closed. The quick-release fastener (40) preferably comprises a magnet that magnetically pulls the quick-release fastener (40) into a closed position and locks the fastener in the closed position. As a result, the quick-release fastener (40) resists inadvertent opening of the quick-release fastener. In order to set the length of the loop, an end of the end belt (9, 10, 11, 12) that is arranged on the upper belt portion is fastened, by means of a hook and loop connection (18, 19, 20, 21), to the lower end of the end belt (9, 10, 11, 12) in a region that can be selected for the purpose of length adjustment of the loop.

In one embodiment, the ends of all the upper belt portions are fixed in each case to an associated lower belt portion by means of a hook and loop connection; for example, the end of an upper belt portion (38) is fixed to a lower belt portion (37) by means of a hook and loop connection (39). The loop is thus closed, a quick-release fastener (40) providing an additional possibility for opening or closing the loop, but without releasing the hook and loop connection (39). For this purpose, the quick-release fastener (40) comprises closure elements that can catch in one another and that are held against one another, in a secured manner, by means of a magnet. In addition, a tensioning loop (41) may be provided, which, when tightened, provides assistance prior to the catch fastening.

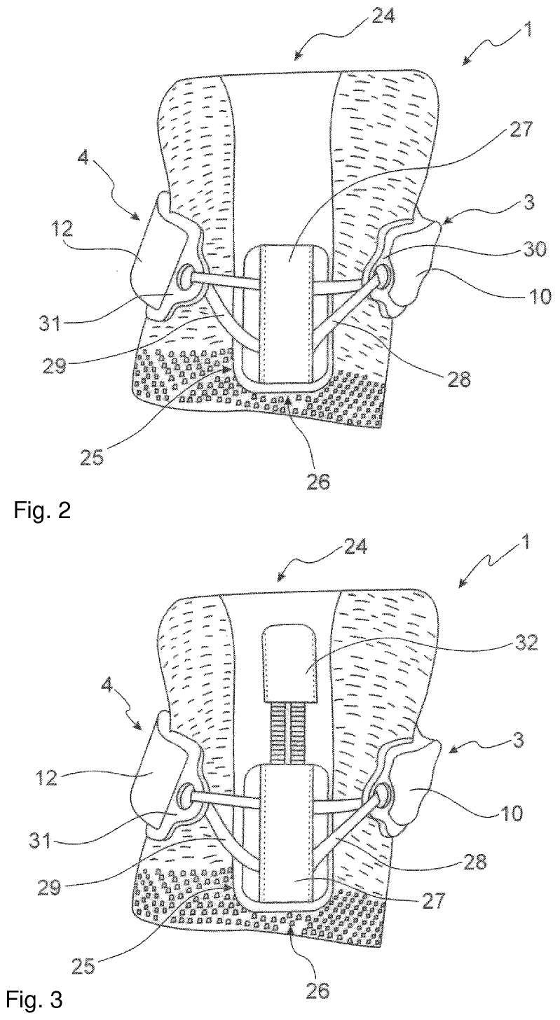

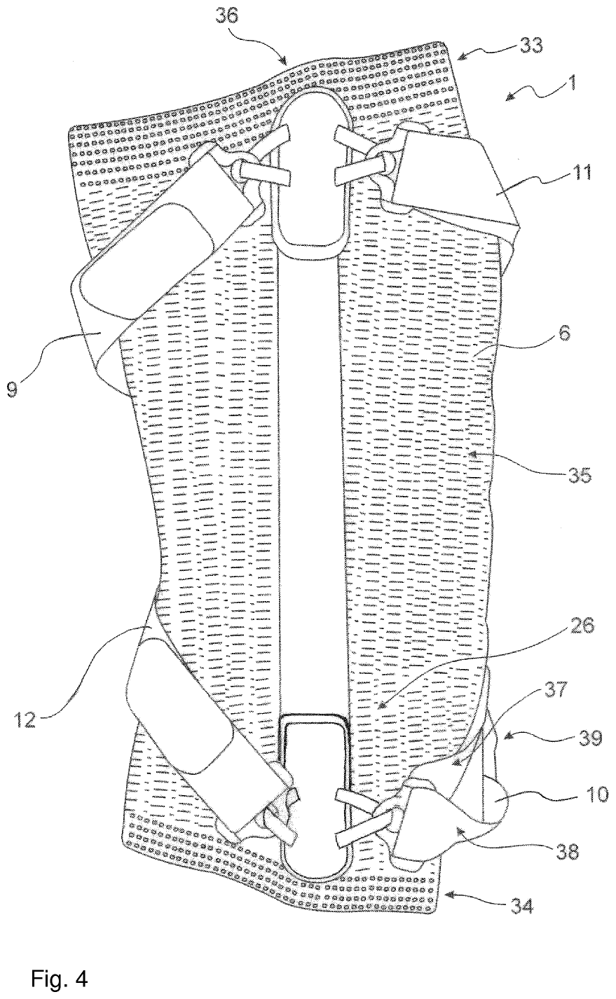

According to one embodiment, the orthosis (1) comprises a cuff (6) on which the belt system is fastened, as can be seen in FIG. 1 and FIG. 2 for example. In this case, the belt system consists of exactly one first belt device (3) comprising a first connection belt (7), and exactly one second belt device (4) comprising a second connection belt (8). Furthermore, in this case, the two belt devices (3, 4) cross over one another in the region of the criss-cross guide (5) and are retained in this region by means of on the cuff (6). In a further embodiment, one belt device (3, 4) is fastened at a proximal region and at a distal region of the cuff (6) in each case. Furthermore, in one embodiment the belt system comprises fastening devices having cords (28, 29), by means of which the two belt devices (3, 4) are fastened to the cuff (6). The cords (28, 29) are in particular strips or strip-like connections. Furthermore, according to this embodiment, the belt system comprises return eyelets on which the end belts of the belt devices are retained, each cord (28, 29) of each fastening device (26) in each case passing through a return eyelet (30, 31) and retaining the return eyelet (30, 31) on the relevant fastening device (26, 36) so as to be rotatable. As is shown for example in FIG. 4, in a preferred embodiment each end belt (9, 10, 11, 12) in each case comprises a lower belt portion and an upper belt portion, a loop being formed in each case by the belt portions, which loop is guided through the relevant return eyelet (30, 31).

The cuff (6) is essentially manufactured from a flexible textile material, and is preferably formed of a woven fabric, a knitted fabric or a warp-knitted fabric. In a particularly preferred embodiment, the material is a resilient material. The orthosis (1) is therefore very easy and comfortable to wear. As shown in FIG. 1, in one embodiment stabilization elements (23, 24), in the form of splints, extending in the proximal-distal direction, are incorporated medially and laterally in the cuff (6). The stabilization elements provide support in the proximal-distal direction, but allow for bending of the knee in the vertical-dorsal direction for example, owing to flexibility or by means of at least one joint. The stabilization elements (23, 24) absorb the vertical components of the belt forces that are introduced, and thus the bandage or orthosis (1) is not compressed. The stabilization elements may be a flexible plastics splint, a flat spiral spring and/or a metal splint. In a preferred embodiment, a first stabilization element (23) is incorporated in the cuff (6) in the proximal-distal direction, as shown in FIG. 1. The first stabilization element (23) is in particular a flexible plastic splint or a flat spiral spring. The criss-cross guide (5) is arranged approximately over a central region of the first stabilization element (23). On a side of the orthosis (1) which is opposite the first stabilization element (23), a second stabilization element (24), in particular in the form of a metal splint comprising at least one joint, is incorporated into the cuff, as can be seen for example in FIG. 2. The second stabilization element (24) preferably extends in the proximal-distal direction and is particularly preferably covered by a loop strip on which a distal fastening device (26) is fastened by means of a hook and loop connection (25). The fastening device (26, 36) may be fastened on the cuff (6) so as to be vertically displaceable. The arrangement of the belt devices (3, 4) can be further optimized thereby. In particular, the orthosis comprises exactly two fastening devices (26, 36) which are both fastened on the cuff (6) so as to be vertically displaceable.

In one embodiment, the at least one fastening device (26, 36) comprises a base part (27) on which each cord (28, 29) of the relevant fastening device (26, 36) is retained at two vertically mutually spaced points in each case and, together with the base part (27), forms a loop, as can be seen in FIG. 2 for example. The two fastening devices (26, 36) preferably each comprise a base part (27). In a particularly preferred embodiment, the base part (27) comprises a hook strip, and the cuff (6) comprises a loop strip, at least in regions; see for example FIG. 2. In this case, the loop strip is preferably a velour surface on the cuff (6). In this case, the hook strip and the loop strip together form a hook and loop connection, by means of which the base part (27) can be fastened and positioned on the cuff (6), in particular is fastened and positioned thereon. The fastening devices (26, 36) can therefore be removed from the cuff (6) if necessary.

Particularly preferably, the points at which a cord (28, 29) of a fastening device (26, 36) is retained are in each case arranged on the base part (27) so as to be paired with the points at which the other cord (28, 29) of the fastening device is retained. In this case, points arranged in pairs are in each case at the same proximal or distal position on the cuff (6). This provides a force equilibrium.

In an advantageous embodiment, each cord (28, 29) of at least one fastening device (26, 36) is guided through the base part, proceeding from one of the vertically mutually spaced points in each case, and, in order to tension the relevant belt device (3, 4), one end of the cord is guided out of the base part (27), as can be seen in FIG. 3 for example. In this case, the relevant cord (28, 29) is preferably deflected in the base part (27) by an angle of between 80° and 100°, in particular between 85° and 95°. It is furthermore preferable for the relevant fastening device (26, 36) to comprise a grip part (32) that is assigned to the base part (27) and that is connected to the ends of the cords of the relevant fastening device (26, 36) that are guided out of the base part. Ends of the cords thus extend approximately in parallel, from the base part (27) to the grip part (32). The two cords can therefore be tightened or loosened together, by means of the grip part (32), in order, if necessary, to change the length of the loops formed by the cords (28, 29). In order to fix the length, the grip part (32) is a gripping tab or a ring and is detachably fixed to the cuff (6), in particular by means of a hook and loop connection. For this purpose, the gripping tab in turn preferably comprises a hook strip, and the cuff (6) comprises a loop strip, as can be seen in FIG. 3 for example.

In one embodiment, a ring is arranged inside the orthosis (1). The ring forms a contact region (35) for the kneecap, as a result of which the position of the orthosis (1) with respect to the kneecap can be better positioned, and the wearing comfort of the orthosis (1) is improved. The contact region (35) is located so as to be approximately central on the cuff (6), as can be seen for example in FIG. 4. The ring or the contact region (35) is located on the inside with respect to the orthosis (1), and therefore the ring is arranged on the orthosis (1) in a covered manner.

This and other embodiments of the present invention are disclosed in the description, the figures and the examples, and are comprised thereby. The features mentioned in the present description and in the claims can, moreover, be combined in any desired selection, and the combinations of features that are expedient within the scope of the invention are to be considered disclosed. Further literature regarding known materials, methods and applications that can be used in accordance with the present invention can be found in public libraries and databases, for example using electronic apparatuses. More complete understanding of the invention can be achieved with reference to the figures and the examples, which have been provided for illustrative purposes and are not intended to limit the scope of the invention.

### EXAMPLES

### Example 1: Structure of an Orthosis for Gonarthrosis, by Way of Example

An embodiment of the orthosis (1) according to the invention, given by way of example, is shown in FIG. 1, FIG. 2 and FIGS. 4 and 5.

In FIG. 1, the orthosis (1) comprises a belt system (2) having a first belt device (3), a second belt device (4), a criss-cross guide (5) for the belt device (3, 4), and a cuff (6). The belt devices (3, 4) cross over one another in the region of the criss-cross guide (5) and are retained on the cuff (6), in this region, by means of the criss-cross guide (5). In this case
(a) the first belt device (3) comprises
   i) a first connection belt (7);
   ii) a proximal first end belt (9); and
   iii) a distal first end belt 10; and
b) the second belt device (4) comprises
   i) a second connection belt (8) which crosses over the first connection belt (7) in the region of the criss-cross guide (5);
   ii) a proximal second end belt (11); and
   iii) a distal second end belt (12).

The first connection belt (7) comprises a proximal first end region (13), by means of which the connection belt is connected to the proximal first end belt (9) so as to be adjustable with respect to the angle and so as to be detachable. The first connection belt (7) furthermore comprises a distal first end region (14), by means of which the connection belt is connected to the distal first end belt (10) so as to be adjustable with respect to the angle and so as to be detachable.

The second connection belt (8) correspondingly comprises a proximal second end region (15) and a distal second end region (16). The proximal second end region (15) connects the connection belt (8) to the proximal second end belt (11), the connection being adjustable with respect to the angle and being detachable. The distal second end region (16) allows for a corresponding connection of the connection belt (8) to the distal second end belt (12), which connection is also connected so as to be adjustable with respect to the angle and so as to be detachable.

In this case, the first connection belt (7) consists of a first belt element (17), consisting in each case of a foldable tab at each end of the belt element (17), i.e. in the proximal first end region (13) and in the distal first end region (14), and consisting of hook strips in the end regions (13, 14), both on the belt element (17) and on the foldable tab. In this case, the hook strips at the relevant end region (13 or 14) face one another and retain loop strips of the first end region (9, 10) on both sides, which loop strips are in each case provided in the form of a double-sided velour surface of the end belts (9, 10). The hook strips and the loop strips provide a proximal first hook and loop connection (18) and a distal first hook and loop connection (19) between the first end belt (9, 10) and the first connection belt (7). In a corresponding manner, the second connection belt (8) comprises a second belt element (20), and a proximal second hook and loop connection (21) and a distal second hook and loop connection (22) are provided between the second connection belt (8) and the second end belt (11, 12).

In the proximal-distal direction, the cuff (6) comprises a first stabilization element (23) that is incorporated therein, the stabilization element preferably being a flexible plastic splint or a flat spiral spring. The criss-cross guide (5) is arranged approximately over a central region of the first stabilization element (23).

FIG. 2 is a further view of the orthosis (1) according to the invention, specifically the side opposite the first stabilization element (23). On this side, a second stabilization element (24) is incorporated into the cuff (5). The second stabilization element (24) preferably consists of a metal splint comprising at least one joint. The stabilization element (24) extends in the proximal-distal direction and is preferably covered by a loop strip on which a distal fastening device (26) is fastened by means of a hook and loop connection (25).

A base part (27) is arranged on the distal fastening device (26), the back of which base part is provided with a hook strip, the hook and loop connection (25) being formed by means of the hook strip and the above-mentioned loop strip. By means of the hook and loop connection (25), the base part (27) can be fastened at various points on the cuff (6) in the proximal-distal direction.

The distal fastening device (26) further comprises cords (28, 29), or alternatively a common cord. The cords (28, 29) are fastened to the base part (27) at both ends, so as to be mutually spaced. In this case, the cords (28, 29) are arranged in a mutually corresponding manner, such that a force equilibrium is established. The cord (28) is guided through a distal first return eyelet (30) on which the first belt device (3) is retained by means of the distal first end belt (10). The cord (29) is guided through a distal second return eyelet (31) on which the second belt device (4) is retained by means of the distal second end belt (12).

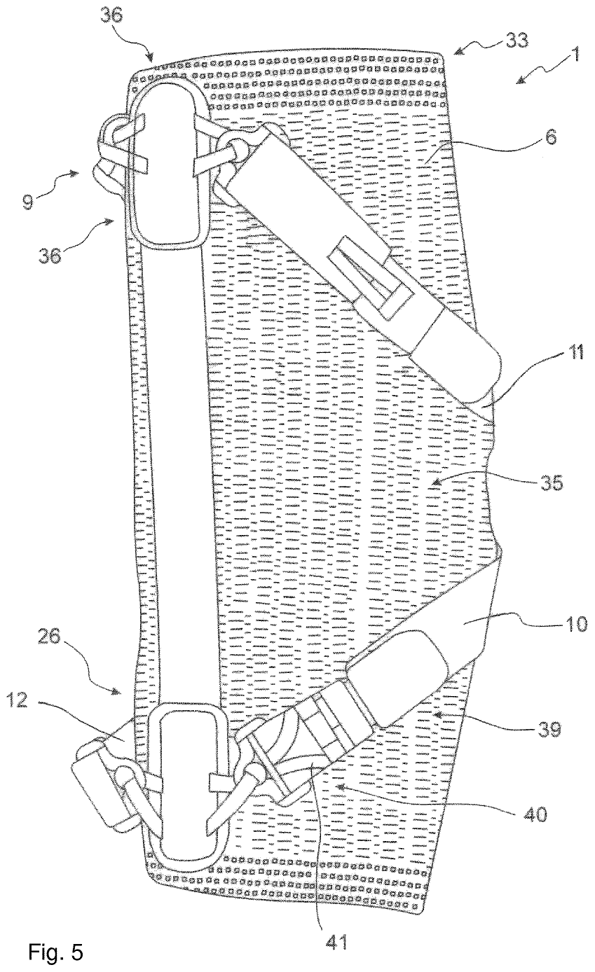

In addition to the distal fastening device (26) in the distal region (34) of the cuff (6), a correspondingly designed proximal fastening device (36) is provided in the proximal region (33) of the cuff (6). This can be seen for example in FIG. 4 and FIG. 5, which figures show the entire proximal-distal extension of the orthosis (1), in two views, specifically from a proximal region (33) of the cuff (6) to a distal region (34) of the cuff (6). The two belt devices (3, 4) are thus retained on both fastening devices (26, 36).

Furthermore, each end belt (9, 10, 11 and 12) in each case comprises a lower belt portion and an upper belt portion, a loop being formed in each case by the belt portions, which loop is guided through the relevant return eyelet (30, 31). For reasons of clarity, in FIG. 4 only the lower belt portion of the distal first end belt (10) is denoted by reference sign (37), and the upper belt portion by reference sign (38). The ends of all the upper belt portions are fixed in each case to the associated lower belt portion by means of a hook and loop connection; for example, the end of the upper belt portion (38) is fixed to the lower belt portion (37) by means of a hook and loop connection (39). The loop is thus closed, a quick-release fastener (40) providing an additional possibility for opening and closing the loop without having to release the hook and loop connection (39). For this purpose, the quick-release fastener (40) comprises closure elements that can catch in one another and that are held against one another, in a secured manner, by means of a magnet. In addition, a tensioning loop (41) is provided, which, when tightened, provides assistance prior to the catch fastening.

Furthermore, as shown in FIGS. 4 and 5, a contact region (35) for the kneecap, in the form of a covered ring, is formed approximately in the center of the cuff (6).

### Example 2: Orthosis Comprising an Alternative Cord Fastening

In the alternative embodiment which is shown by way of example in FIG. 3, the cords (28, 29) are guided through the base part (27), on the proximal side, as far as a distal grip part (32), and are not fastened to the base part (27). The grip part (32) makes it possible to change the length of loops that are formed by the cords (28, 29) and the base part (27). In this case, the distal grip part (32) may be fixed to the cuff (6) on a velour surface, by means of a detachable hook and loop connection.

### Example 3: Orthosis Comprising a Fixed Cord Fastening

In one embodiment of the present orthosis according to the invention, the orthosis comprises fixed cord fastenings. For this purpose, the base parts (27) are rigidly connected to the pocket of the stabilization element (24) at the proximal and distal ends.

1-15. (canceled)

16. Orthosis for gonarthrosis, for relieving the stress on the medial or lateral knee compartment of the human knee, the orthosis comprising:
a) a belt system, the belt system including at least one tension-resistant belt device;
b) each of the at least one belt device having at least one end belt;
c) each of the at least one belt device having at least one connection belt, and which connection belt is connected to each end belt of the relevant belt device so as to be angularly adjustable.

17. Orthosis according to claim 16, wherein:
a) the belt system includes a criss-cross guide for two belt devices, the two belt devices crossing over one another in the region of the cross-cross guide, and being retained in a positive manner by means of the criss-cross guide.

18. Orthosis according to claim 16, wherein:
a) the at least one end belt of the at least one belt device includes, in each case, exactly one proximal end belt of the relevant belt device and exactly one distal end belt of the relevant belt device, and the connection belt of the relevant belt device includes a proximal end region, by means of which the relevant connection belt is connected to the proximal end belt of the relevant belt device, and a distal end region, by means of which the relevant connection belt is connected to the distal end belt of the relevant belt device.

19. Orthosis according to claim 16, wherein:
a) at least one end belt of at least one belt device is connected to the connection belt of the relevant belt device by means of a hook and loop connection, so as to be angularly adjustable and so as to be detachable.

20. Orthosis according to claim 19, wherein:
a) the connection belt of the relevant belt device includes a belt element having a front face and a rear face, and in each case has, at each end of the belt element, on the rear face thereof, a hook strip for the hook and loop connection, and a foldable tab that is arranged so as to correspond to the hook strip and has a further hook strip facing the hook strip, in that the end belts include a loop strip on both sides, at least in portions, which loop strip can be positioned between the hook strips, and in that the end belts can be connected to the connection belt so as to be adjustable with respect to the angle, by means of the hook and loop connection that is provided by the hook strips and the loop strips.

21. Orthosis according to claim 16, wherein:
a) at least one end belt of at least one belt device is connected to the connection belt of the relevant belt device, by means of a rivet connection, so as to be adjustable with respect to the angle.

22. Orthosis according to claim 16, wherein:
a) for the purpose of length adjustment, at least one end belt of at least one belt device includes a lower belt portion and an upper belt portion which is wrapped around on the lower belt portion, and a length-adjustable loop being formed by the belt portions.

23. Orthosis according to claim 22, wherein:
a) the belt device includes a quick-release fastener on the upper belt portion of the end belt, by means of which quick-release fastener the loop is releasably closed, the quick-release fastener preferably including a magnet that magnetically pulls the quick-release fastener into a closed position and locks the fastener in the closed position.

24. Orthosis according to claim 22, wherein:
a) an end of the end belt that is arranged on the upper belt portion is fastened, by means of a hook and loop connection, in a region that can be selected for length adjustment of the loop.

25. Orthosis according to claim 16, wherein:
a) the orthosis includes a cuff on which the belt system is fastened, the at least one belt device includes exactly one first belt device having a first connection belt, and exactly one second belt device including a second connection belt, in that the two belt devices cross over one another and are each fastened at a proximal region and at a distal region of the cuff, in that the belt system includes fastening devices having cords, by means of which the two belt devices are fastened to the cuff, and in that the belt system includes return eyelets on which the end belts of the belt devices are retained, and each cord of each fastening device in each case passing through a return eyelet and retaining the return eyelet on the relevant fastening device so as to have one translational degree of freedom and two rotational degrees of freedom.

26. Orthosis according to claim 25, wherein:
a) at least one fastening device is fastened on the cuff so as to be vertically displaceable.

27. Orthosis according to claim 25, wherein:
a) at least one fastening device includes a base part on which each cord of the relevant fastening device is retained at two vertically mutually spaced points in each case and, together with the base part, forms a loop in each case.

28. Orthosis according to claim 27, wherein:
a) the base part includes a hook strip, in that the cuff includes a loop strip at least in regions, and in that the hook strip and the loop strip together form a hook and loop connection, by means of which the base part can be fastened and positioned on the cuff.

29. Orthosis according to claim 27, wherein:
a) the points at which a cord of a fastening device is retained are in each case arranged on the base part so as to be paired with the points at which the other cord of the fastening device is retained.

30. Orthosis according to claim 28, wherein:
a) each cord of at least one fastening device is guided through the base part, proceeding from one of the vertically mutually spaced points in each case, and in the process is deflected in the base part by an angle of between 80° and 100° and, in order to tension the relevant belt device, one end of the cord is guided out of the base part, and in that the relevant fastening device includes a grip part that is assigned to the base part and that is connected to the ends of the cords of the relevant fastening device that are guided out of the base part.
